(19) United States
(12) Patent Application Publication
(10) Pub. No.: US 2004/0068262 A1
(43) Pub. Date: Apr. 8, 2004

(54) Soft tissue fixation implant

(76) Inventors: Mark Lemos, Carlisle, MA (US); Tero Valimaa, Turku (FI); Petri Kujansuu, Ikaalinen (FI); Mika Vihavainen, Tampere (FI); Pertti Tormala, Tampere (FI)

Correspondence Address: KENYON & KENYON, 1500 K STREET, N.W., SUITE 700, WASHINGTON, DC, 20005, US

(21) Appl. No.: 10/261,413

(22) Filed: Oct. 2, 2002

(51) Int. Cl. A61B 17/56

(52) U.S. Cl. 606/72

(57) ABSTRACT

In the present invention, a bioabsorbable implant is described, which may be manufactured of bioabsorbable polymer, copolymer, polymer alloy or fiber reinforced and/or particle filled composite, and which may be pushed into a hole or drill canal made in bone, for fixing a soft tissue graft into the drill hole. The implant of the present invention includes an body having an outer surface which comprises at least one gripping element to lock the implant directly into the drill hole, and at least one recess for receiving soft tissue grafts.

### FIELD OF THE INVENTION

The present invention relates to bioabsorbable surgical implants, which are used to fix tissue grafts to hard tissue, such as bone, and a method for inserting such implants in a patient.

### BACKGROUND OF THE INVENTION

In surgery it is generally known to use soft tissue tendon grafts (e.g. hamstring tendon, taken from the thigh of the patient) to replace the severely damaged anterior cruciate ligament (ACL). In a typical surgical procedure one end of a soft tissue graft is fixed into a drill hole made from the knee joint into the distal femur and another end of the graft is fixed into a drill hole made into the proximal tibia. The ends of the graft are fixed into the drill holes with fixation screws and in most cases with so-called interference screws. A screw is installed into the space between the drill hole and the soft tissue grafts to lock the grafts into the drill hole. The tendon then acts as a new ACL.

The fixation screws, like interference screws, are normally made of metal like stainless steel or titanium, or of a bioabsorbable polymer like polylactide. Metallic and/or bioabsorbable polymeric materials and composites, which are suitable for manufacturing of tendon graft fixation screws, are well known in the art, for example as described in the literature. See. e.g. Weiler A et. al., “The Influence Of Screw Geometry On Hamstring Tendon Interference Fit Fixation”, The American Journal of Sports Medicine, vol. 28, No. 3, 2000, pp. 356-359; Barber. A. F, Burton F. McGuire D. A., and Paulos L., “Preliminary Results Of An Absorbable Interference Screw”, The Journal of Arthroscopic and Related Surgery, vol. 1. No. 5 (1995) at 537-548; and Bach. B. R., “Arthroscopy Assisted Patellar Tendon Substitution for Anterior Cruciate Ligament Insufficiency”, American Journal of Knee Surgery, vol. 2. No. 1 (1989) at 3-20, the disclosures of which are hereby incorporated by reference.

The surgical technique of soft tissue graft fixation is described e.g. in Hoffmann R et al. “Initial Fixation Strength Of Modified Patellar Tendon Grafts For Anatomic Fixation In Anterior Cruciate Ligament Reconstruction”, Arthroscopy, vol. 15, No. 4, 1999, pp. 392-399, the disclosure of which is herein incorporated by reference.

WO 01/30253 discloses an orthopedic ligament fixation system, which comprises a threaded expandable screw, which is expanded by insertion of an expansion screw into a threaded longitudinal bore of the expandable screw. The expandable screw is mounted in a rotating manner. However, this implant is complicated because of its two-piece structure, tedious procedure of rotating the screw, and time consuming threading of the drill-hole.

U.S. Pat. No. 5,906,632 discloses an intratunnel attachment device. First, a deformable ring is inserted in a sliding manner into a bone tunnel, and after that a screw is inserted by rotating the screw inside the ring. However, this is a complicated, two-piece implant, and the mounting of the device is done with rotation.

U.S. Pat. No. 5,935,129 discloses an apparatus for anchoring objects, such as soft tissue to bone. This apparatus comprises an anchoring element, and an expander element. The expander element is pulled through the anchoring element causing expanding of the anchoring element, and hence attaching the outer surface of the anchoring element to the walls of the bone tunnel. This is also a complicated two-piece implant.

These conventional extra-articular hamstring graft fixation techniques have complications, such as suture stretch, graft tunnel motion and so-called windshield wiper effect where the size of the intra-articular drill hole end will increase due to graft movement in the drill-hole. Also the use of screws as fixation implants for soft tissue grafts in anterior crucial ligament procedures is complicated due to: 1) the threads of the screw cutting the grafts during screw installation if the screw is too big in relation to the tendon and/or if the space between the drill hole and tendon grafts is too small; 2) the threads of the screw damaging the tendon during screw installation; 3) the tendon rotating with the screw during screw installation so that the optimal position of the grafts is lost and/or the grafts are damaged; 4) divergence of the grafts and/or screw occurring; and 5) the bioabsorbable screw breaking during insertion.

Accordingly, there is a need for a simple, preferably one-piece, soft tissue fixation implant, which is pushed into a drill-hole in a bone to lock a soft tissue graft, like a tendon or ligament graft, into the drill-hole rapidly and effectively with minimal risk of damaging the soft tissue graft during insertion.

### SUMMARY OF THE INVENTION

The present invention provides a surgical implant for securing a tissue graft to hard tissue. In one embodiment of the present invention, a bioabsorbable implant for securing a tissue graft in bone is provided, including a body having an external perimeter, where the external perimeter includes at least one gripping element configured to secure the implant in the bone, and further where the external perimeter includes at least one recess configured to receive the tissue graft, located longitudinally along the implant body.

Another embodiment of the present invention includes a bioabsorbable implant for securing a tissue graft in bone, having a forked body including at least two prong portions and a transverse ridge connecting the two prong portions, where the forked body has an external perimeter. And further where said external perimeter includes at least one gripping element configured to secure the implant in the bone, and further where the external perimeter includes at least one recess configured to receive the tissue graft, located longitudinally along the forked body.

In yet another embodiment of the present invention, a method for securing at least one tissue graft to bone is provided. The method includes the steps of attaching at least one tissue graft to a bioabsorbable implant, where the implant includes a body, at least one gripping element on an outside surface of the implant body configured to secure the implant in the bone hole, and at least one recess configured to receive the tissue graft, located longitudinally along the implant body, inserting the bioabsorbable implant and the tissue graft in a bone hole, and securing the bioabsorbable implant in the bone hole by the gripping element.

### BRIEF DESCRIPTION OF THE DRAWINGS

FIGS. 1a-c show longitudinal cross-sections of embodiments of the implant of the present invention.

FIG. 2 shows a perspective view of an embodiment of the implant of the present inventon.

FIGS. 3a-b illustrate side views of embodiments of the implant of the present invention provided with tissue holding means.

FIGS. 4a-d show cross-sectional views of embodiments of the implant of the present invention provided with different expanding elements.

FIGS. 5a-c show perspective views of distal ends of embodiments of the present invention together with installation instruments.

FIGS. 6a-b show perspective views of embodiments of the present invention including soft tissue grafts located over the implant.

FIG. 7 shows a cross-sectional view of an embodiment of an implant of the present invention inserted in a bone hole.

### DETAILED DESCRIPTION OF THE PREFERRED EMBODIMENTS

The present invention provides a bioabsorbable implant for securing a tissue graft in bone or other hard tissue. Like numbers shall be used for like elements throughout the Figures.

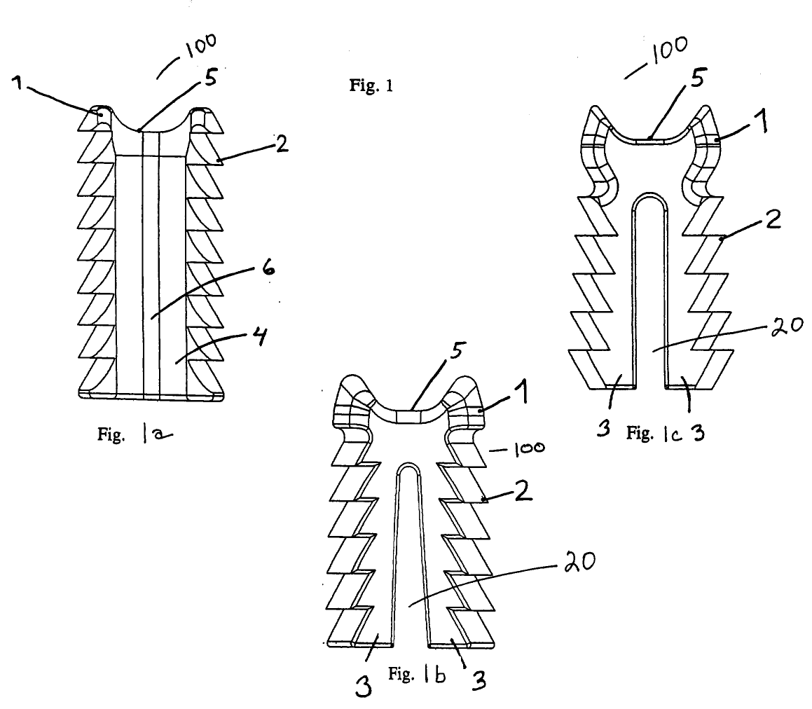

As shown in FIGS. 1a-c, the implant 100 comprises a implant body 1 and at least one gripping means 2 along a portion of the external perimeter of the implant body 1. These gripping means 2 may be any type of element or protuberance, such as barbs, scales, threads, pyramid formations, ridges, or combinations thereof. The gripping means 2 allows the implant 100 to be easily attached to bone, but makes it difficult to remove as the gripping means 2 engage firmly the surface of the bone following fastening. The implant 100 may be inserted into hard tissue through a void in the hard tissue. The hard tissue is preferably bone and the void can be any void within hard tissue, such as a drill hole, canal, cavity, groove, hollow, opening, or the like.

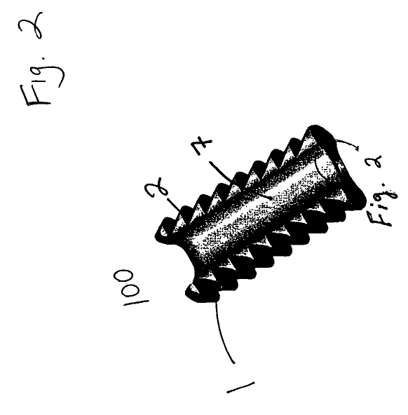

The implant 100 also includes at least one recess 7, shown in FIG. 2. This recess 7 runs along a longitudinal axis of the implant body 1. The recess 7 is configured to receive a tissue graft 8 (not shown in FIG. 2). The recess 7 may be a depression, channel, trough, or the like. The recess 7 may have shallow forms instead of sharp edges because the resulting shear forces from sharp edges are highly undesirable as they may tear or damage the tissue graft. Therefore, the outer surface or perimeter of the implant body 1, outside of the recess 7, is preferably where the gripping elements 2 or located.

In one embodiment of the present invention, shown in FIG. 1a, the implant body 1 is a single piece of material 4. In another embodiment of the present invention, shown in FIGS. 1b-c, the implant body 1 comprises a forked structure, including at least two prong portions 3 and a transverse ridge 5 connecting the two prong portions 3. The outer surface of ridge 5 is preferably concave, thus making it possible to place tissue grafts over ridge 5.

The prong portions 3 and the ridge 5 may form a V-shaped structure, which is compressible during insertion so that the free ends of the prong portions 3 are movable towards each other. Or alternatively, it is also possible that the prong portions 3 may be parallel to one another. The void 20 separating the two prong portions 3, may be configured to accept or receive an insertion tool (not shown).

Additionally, at lest one cavity 6, shown in FIG. 1a may be included in the implant body 1. Cavity 6 is internal to implant body 1 and runs along a longitudinal axis from the distal end of the implant 100 to the insertion end of the implant 100. Cavity 6 may extend through the entire implant 100 or a portion thereof. Additionally, the shape of the cavity may vary, for example, the cavity may be round, oblong, rectangular, or the like. Also, while the embodiment shown with a cavity in FIG. 1 a is a solid body 1, cavities may also be located in one or more of the prong portions 3 shown in FIGS. 1b-c.

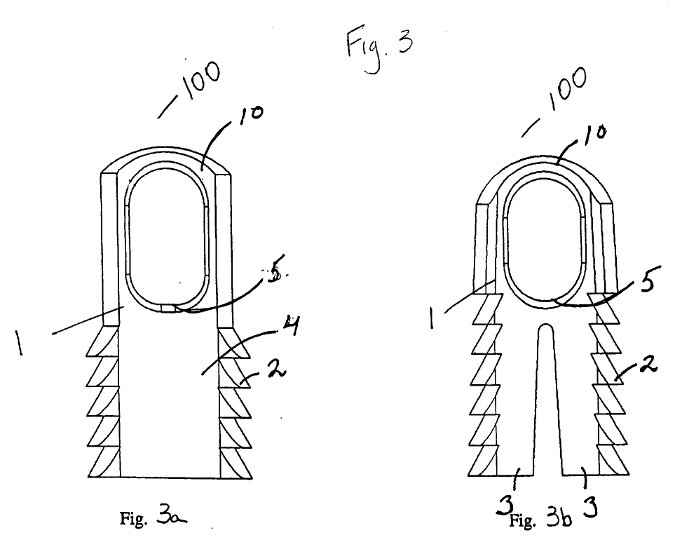

According to FIGS. 3a-b, the implant body 1 can further comprise means 10 for holding a tissue graft to the implant 1. The means may be located at the insertion end of the implant 100 and may be any structure containing a cutout, gap, notch, opening, slot, hole, or the like, which will hold or secure a tissue graft to the implant body 1. The shape of the cutout may preferably be circular or oblong to reduce rough edges. The holding means may also be used with any type of implant 100, such as ones having a solid body or a forked body. The holding means effectively prevents the slipping of a tissue graft from the implant 100 during and after insertion into a bone, and protects the tissue graft from touching the surfaces of the bone.

In an embodiment of the present invention, the holding means may be a closed loop 10 located above the concave ridge 5 of the implant 100. Closed loop 10 may act as a protective shield to a tissue graft and therefore, during insertion of the implant 100 with closed loop 10 the tissue graft is kept in place so that no slipping occurs.

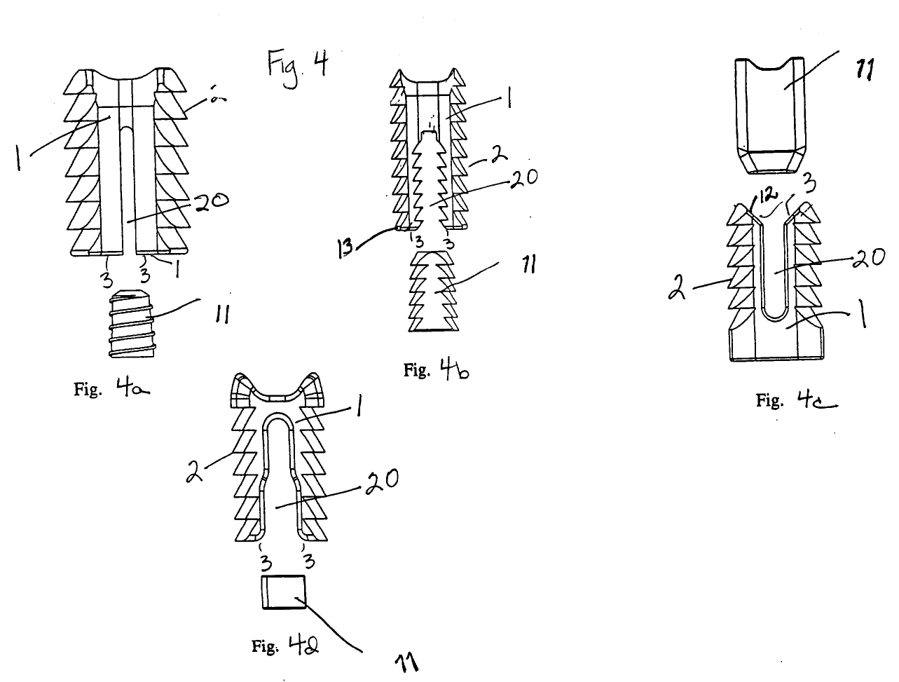

In another embodiment of the present invention shown in FIGS. 4a-d, the implant body 1 may be expanded by an expanding element 11. The expansion of implant body 1, occurs following insertion into bone. Once the implant body 1 is inserted into bone, expanding element 11 may be pushed inside the implant body 1 to enhance fastening the implant 100 to the bone. The expanding element 11 is received in the implant 100, through the void 20 formed by prong portions 3 of the implant body 1. The inner surfaces of the prong portions 3 forming the void 20 may be textured in order to enhance receiving the expanding element 11. For example, the inner surfaces of the prong portions 3 may include protrusions, threads, barbs, scales, or the like. Also the expanding element 11 may be preferably conical, such as a conical screw, or other element that may be turned inside the implant body 1 to expand it in the bone.

The bioabsorbable implant 100 and the expanding element 11 of the invention may be manufactured of bioabsorbable (biodegradable or resorbable) polymers, copolymers, polymer alloys or composites, e.g., of poly-alpha-hydroxide acids and other aliphatic biodegradable polyesters, polyanhydrides, polyorthoesters, polyorganophosphatenes, and other bioabsorbable polymers disclosed in numerous publications, e.g., in Finnish Patent Application No. FI-952884 (corresponding publication U.S. Pat. No. 6,007,580) and FI-955547 (corresponding publication GB 2307179), and PCT Application No. WO-90/04982, the disclosures of which are herein incorporated by reference.

The implants of the present invention may also be reinforced by reinforcing material such as fibers manufactured of resorbable polymer or polymer alloy, or biodegradable glass fibers, such as β-tricalciumphosphate fibers, bio-glassfibers or CaAl fibers (cf., e.g., European Patent Application No. EP146398, the disclosure of which is herein incorporated by reference). Ceramic powders can also be used as additives (fillers) in the implants of the present invention, to promote new bone formation.

In addition, the implants of the present invention may also be formed from layers including, e.g., (a) a flexible surface layer, which may improve the toughness of the implant, be used for releasing drugs or other bioactive substances, and/or may act as a hydrolysis barrier, and (b) a stiff inner layer.

In other embodiments of the present invention, the implants may also contain various additives for facilitating the processability of the material (e.g., stabilizers, antioxidants or plasticizers), for changing its properties (e.g., plasticizers or ceramic powder materials or biostable fibers, such as carbon fibers), or for facilitating its treatment (e.g., colorants).

In another embodiment of the present invention, the implant (and/or its surface layer) may contain bioactive agent or agents, such as antibiotics, chemotherapeutic agents, agents activating healing of wounds, growth factor(s), bone morphogenic protein(s), anticoagulant (such as heparin), etc. Such bioactive implants are particularly advantageous in clinical use, because they have, in addition to their mechanical effect, also biochemical, medical and other effects in various tissues.

The present invention may also include embodiments of the implant having holes or open porosity to facilitate tissue or bone growth inside of the implant. Such holes or pores typically have a diameter from 100 μm to 2000 μm. The holes or pores may be filled with cancellous bone of the patient, or with ceramic bone substitute powder or granules (e.g., bioglass), to accelerate their filling with new bone. Such new bone inside of holes or pores of the implant facilitates the final healing of a drill hole and the fixation of the soft tissue grafts inside of the drill hole when the implant biodegrades and disappears from the drill hole.

Surgical implants in accordance with the invention may be manufactured of biodegradable polymers and of suitable biodegradable reinforcement fibers by means of various methods used in plastic technology, such as injection molding, extrusion and fibrillation, and molding related thereto (cf, e.g., U.S. Pat. No. 4,968,317, the disclosure of which is herein incorporated by reference). Or by means of compression molding, wherein the implant pieces are shaped from the raw material by employing heat and/or compression. Also mechanical machining (e.g. cutting, drilling, lathing, grinding etc.) may be used.

It is also possible to manufacture implants of the present invention using the aforementioned polymeric raw materials and dissolving at least part of the polymer in a suitable solvent or softening the polymer by means of that solvent, and then compressing the polymer into an implant piece by means of pressure and/or by means of slight heat, wherein the dissolved or softened polymer is glued to form a macroscopic implant piece wherefrom the solvent is removed by evaporation.

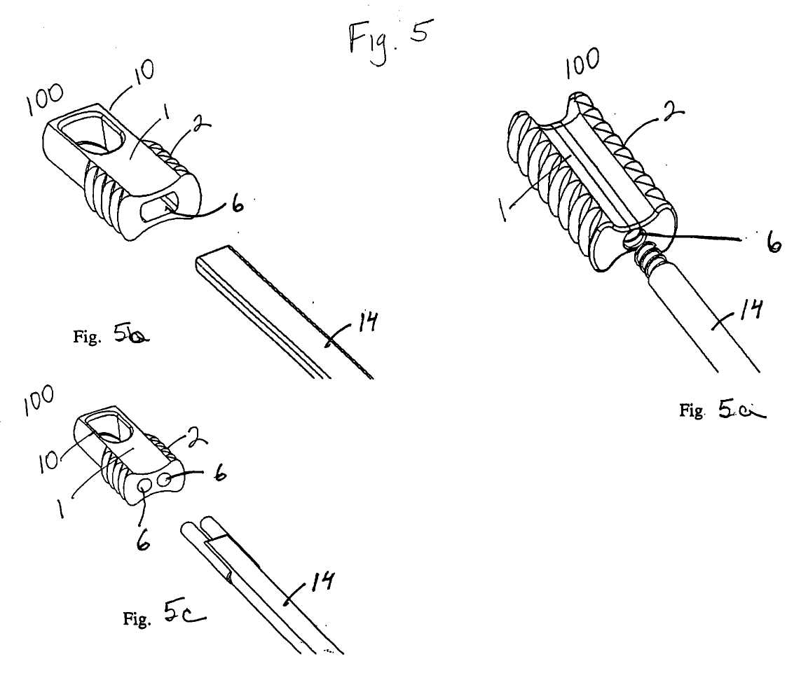

The implant 100 of the present invention may be used with an insertion tool 14, as shown in FIGS. 5a-c. Insertion tool 14 may be inserted or received into the implant 100 through cavity 6. The shape and the size of the tip of the insertion tool 14 and the cavity 6 may vary, however the tip of the insertion tool 14 and the cavity 6 should complement each other. For example, as shown in FIG. 5a, cavity 6 has a smooth rectangular shape as does the tip of the insertion tool 14. FIG. 5b shows cavity 6 having a circular threaded shape as does the tip of insertion tool 14. Finally, FIG. 5c shows implant 100 having two cavities 6 and the tip of the corresponding insertion tool 14 having a split end. The insertion tool 14 may have a depression similar to depression 7 on implant 100 to allow the tissue graft to rest along it.

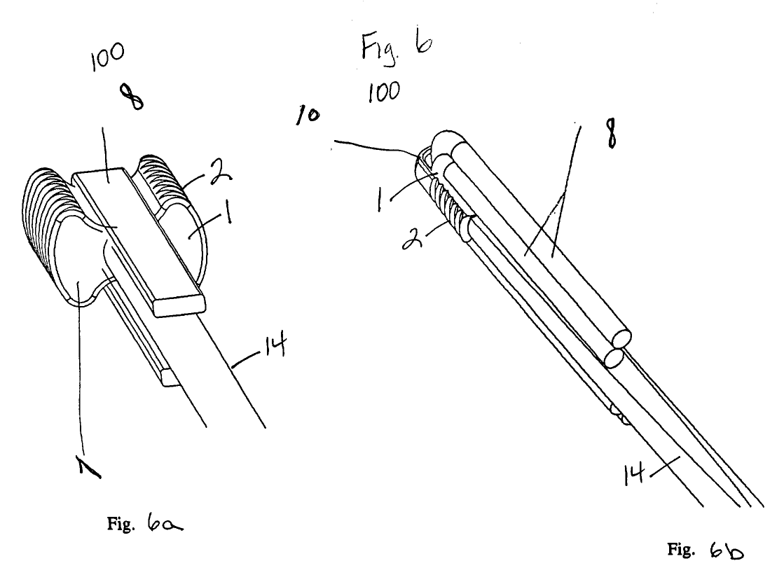

FIGS. 6a-b show tissue grafts 8 placed over the implant body 1 and an insertion tool 14. The tissue grafts 8 are located in depression 7 on the implant body 1 and a depression on the insertion tool 14. In addition, in FIG. 6b, the tissue grafts 8 are placed through holding means 10.

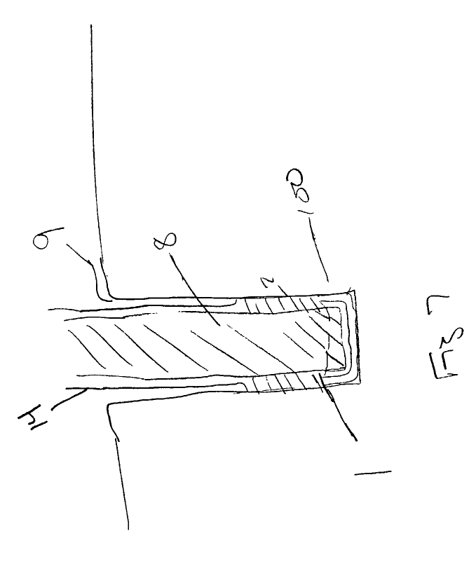

Another embodiment of the present invention, includes a method for securing a tissue graft in hard tissue. To begin with, a hole is made in a hard tissue, such as bone. The hole is preferably formed by a drill. Next, a tissue graft 8 may be preferably attached to an implant 100 prior to insertion into the drill hole, or the tissue graft 8 can be inserted first into the drill hole and then the implant 100 can be inserted. FIG. 7 shows an implant 100 inserted into a bone hole 9. The implant 100 has a tissue graft 8 laying along depression 7 and a depression on insertion tool 14.

In the insertion process, the implant is mounted in a sliding manner so that it may be pushed inside the drill hole made in the bone. The insertion process is preferably conducted using insertion tool 14 with which the implant 100 can be taken into a firm grip, inserted into the drill hole, and released easily after insertion.

If the implant does not use an expanding element 11, the implant may be compressed prior to insertion and then released after the insertion process. Thus the outer surface of the implant body touches the inner surface of the bone hole following release. In addition, the gripping elements 2 start to straighten after the insertion process. If the implant does use an expanding element 11, the expanding element 11 causes the prong portions 3 to expand against the inner surface of the bone hole 9.

A tight fit of the implant 100 and tissue graft 8 into the drill hole 9 is achieved when the maximum thickness of the implant 100 combined with the maximum thickness of the tissue graft 8 is greater than the diameter of the drill hole 9. All the edges contacting the tissue graft are preferably rounded to prevent irritation of the tissue graft.

Thus, by applying implants according to the invention it is possible to efficiently attach and immobilize soft grafts into drill holes in bone, against forces tending to loosen the grafts, without having to carry out a time-consuming and risky fixation with a screw, which may damage the soft tissue grafts.

The invention is not restricted to what has been described above and shown in the drawings, but can be modified and supplemented in many different ways within the scope of the invention defined in the claims.

What we claim is:

1. A bioabsorbable implant for securing a tissue graft in bone, comprising:
a body having an external perimeter,
wherein said external perimeter includes at least one gripping element configured to secure the implant in the bone, and
further wherein said external perimeter includes at least one recess configured to receive the tissue graft, located longitudinally along said body.

2. The implant of claim 1, wherein the gripping element is selected from the group consisting of protrusions, barbs, scales, ridges, or combinations thereof.

3. The implant of claim 1, further having at least one internal cavity configured to receive an insertion tool.

4. The implant of claim 3, wherein the implant has two internal cavities.

5. The implant of claim 1, wherein the body is porous.

6. The implant of claim 1, wherein the body includes at least one bioactive substance, which is released over time.

7. The implant of claim 1, further comprising means for holding the tissue graft to the implant.

8. The implant of claim 7, wherein the means for holding include a loop located at an insertion end of the implant.

9. A bioabsorbable implant for securing a tissue graft in bone, comprising:
a forked body including at least two prong portions and a transverse ridge connecting the two prong portions, wherein the forked body has an external perimeter, and
wherein said external perimeter includes at least one gripping element configured to secure the implant in the bone, and at least one recess configured to receive the tissue graft, located longitudinally along said forked body.

10. The implant of claim 9, wherein the two prong portions are capable of being compressed towards each other.

11. The implant of claim 9, wherein the two prong portions are partially separated by a void.

12. The implant of claim 9, wherein the void is configured to receive an expanding element.

13. The implant of claim 9, wherein at lesat one prong portion includes an internal cavity configured to receive an insertion tool.

14. The implant of claim 9, wherein the implant further comprises means for holding the tissue graft to the implant.

15. The implant of claim 14, wherein the means for holding includes a loop attached to the traverse ridge of the implant.

16. A method for securing at least one tissue graft to bone, comprising:
attaching the at least one tissue graft to a bioabsorbable implant, wherein the implant includes a body, at least one gripping element on an outside surface of said body configured to secure the implant in a bone hole, and at least one recess configured to receive the tissue graft, located longitudinally along said implant body;
inserting the bioabsorbable implant and the tissue graft in the bone hole; and
securing the bioabsorbable implant in the bone hole by the gripping element.
